SEQUENCE LISTING (5 sequences)

SEQ ID NO: 1 - DNA, 18 bp, Artificial Sequence
  Probe EUB
gctgcctcccgtaggagt

SEQ ID NO: 2 - DNA, 23 bp, Artificial Sequence
  Probe JZ1
tggcgcttgaagagggcccgcgc

SEQ ID NO: 3 - DNA, 23 bp, Artificial Sequence
  Probe JZ2
cccgggctaacatcccgggagag

SEQ ID NO: 4 - DNA, 42 bp, Artificial Sequence
  PCR primer pair fd1 + rp1
agagtttgatcctggctcagcacggttaccttgttacgactt

SEQ ID NO: 5 - DNA, 35 bp, Artificial Sequence
  PCR primer pair EUB + Y1540R
gctgcctcccgtaggagtaaggaggtgatccagcc

(19) United States
(12) Patent Application Publication
(10) Pub. No.: US 2003/0099928 A1
(43) Pub. Date: May 29, 2003

(54) Method of isolating unculturable microorganisms

(76) Inventor: Robert S. Burlage, Knoxville, TN (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 09/800,853

(22) Filed: Mar. 7, 2001

Related U.S. Application Data
(63) Continuation of application No. 09/397,196, filed on Sep. 16, 1999, now abandoned.

(51) Int. Cl. C12Q 1/70; C12Q 1/68; C07H 21/04

(52) U.S. Cl. 435/5; 435/6; 536/24.3

(57) ABSTRACT

Methods for identifying and isolating an unculturable microorganism in a collection of microorganisms utilizing a nucleic acid sequence complementary to a variable region of the 16S rRNA of the target microorganism. Unculturable microorganisms are identified by comparing the rRNA sequences of the collection of microorganisms to either the rRNA sequences from databases of culturable microorganisms or to the rRNA sequences of cultures of the collection of microorganisms. A target unculturable is selected based upon various criteria. The probe contains a label that enables detection or separation of the labeled microorganism. The DNA can be recovered from the separated microorganism and portions thereof can be cloned into culturable microorganisms to synthesize the gene product.

### STATEMENT OF GOVERNMENT RIGHTS

This invention was made with Government support under Contract No. DE-AC05-96OR22464 awarded by the U.S. Department of Energy to Lockheed Martin Energy Corp., and the Government has certain rights in this invention.

### TECHNICAL FIELD

The invention relates to unculturable microorganisms and more specifically to methods for isolating unculturable microorganisms and of isolating DNA from unculturable microorganisms.

### BACKGROUND OF THE INVENTION

Unculturable microorganisms are those microorganisms that cannot be grown in pure cultures by known means. It has been hypothesized that the majority of microorganisms are unculturable in any microbial environment, for reasons that are unclear. These unculturable microorganisms represent a vast, untapped resource of DNA and of potential products such as enzymes and other protein products. These microorganisms undoubtedly have undiscovered enzymes that would be useful for environmental, medical, or industrial applications. However, these potential products are currently unavailable because there is no known way to obtain pure DNA from such microorganisms.

Microorganisms are presently studied at the genetic level by cloning and sequencing their DNA. However, this requires the cultivation of a large number of cells with subsequent extraction of the DNA from the batch. The same approach obviously can not be used for unculturable microorganisms.

Nucleic acid hybridization is a well known technique for specifically detecting the presence of a nucleic acid even in the presence of a large quantity of other nucleic acids. The technique generally involves the use of a probe comprising a nucleic acid polymer that is complementary to the target nucleic acid. The probe also includes a label that can be visualized or otherwise detected.

E. coli cells contain over 15,000 ribosomes. Ribosomes contain proteins and RNA, termed ribosomal RNA, or rRNA, which comprises about 82% of the RNA in the cell and about 65% by weight of the ribosome. There are three types of rRNA in prokaryotic organisms, 16S, 23S, and 5S. The 16S rRNA has a molecular weight of about 550,000 and about 1500 nucleotides. The 16S rRNA contains variable regions, or nucleic acid sequences that are different for each microorganism, and conserved regions, or regions that are the same for all microorganisms or for species of microorganisms. Some regions of the 16S rRNA are invariant in all organisms and some are unique to particular groups of organisms. rRNA is preferable to DNA as a probe target because of its relative abundance and stability in the cell and because of its patterns of phylogenetic conservation.

Giovannoni, S. J. et al., J. Bacteriology, 170:720-726 (February 1988) teaches the use of hybridization probes specific for each of the three primary lines of evolutionary descent; the eubacteria, the archebacteria, and the eucaryotes. The probes are nucleic acid polymers specific for the conserved regions of the rRNA.

U.S. Pat. Nos. 5,928,864 and 5,693,468 to Hogan et al. also teach nucleic acid probes specific for the conserved regions of rRNA for detecting particular organisms.

Wallner, G. et al., Cytometry 14: 136-143 (1993), and Appl. Envir. Micro. 63: 4223-4231 (1997), also describe the use of nucleic acid probes complementary to the conserved regions of rRNA for identifying and classifying microorganisms. Wallner et al. combined hybridization with quantitation of labelled cells using flow cytometry and with cell separation using fluorescence activated cell sorting (FACS).

While the above references teach the detection of certain types of microorganisms contained within a sample, they do not teach the use of probes based upon the variable region of rRNA, which probes will detect the presence of specific, individual microorganisms rather than classes of microorganisms. They also do not teach a method of isolating specific microorganisms or, more particularly, of isolating unculturable microorganisms.

Accordingly, what is needed are methods for identifying and isolating unculturable microorganisms. What is also needed are methods for isolating DNA from unculturable microorganisms.

### SUMMARY OF THE INVENTION

The present invention is directed to methods for identifying and isolating unculturable microorganisms.

The present invention is also directed to methods for identifying and isolating the DNA from unculturable microorganisms.

The present invention also is directed to methods for producing gene products of unculturable microorganisms.

Furthermore, the present invention is directed to DNA isolated from unculturable microorganisms and to the gene products from such DNA.

The methods for identifying unculturable microorganisms in a collection of microorganisms involve sequencing the rRNA of all of the microorganisms in the sample. The rRNA sequences are then compared against the rRNA sequences from databases of culturable microorganisms. Alternatively, the collection of microorganisms are cultured and the rRNA sequenced and the rRNA of all of the microorganisms in the collection are compared against the rRNA sequences of the cultured microorganisms. The difference in either comparison includes the rRNA of the unculturable microorganisms. A target unculturable is selected based upon various criteria.

A probe is then designed that comprises a nucleic acid sequence complementary to a variable region of the rRNA of the target microorganism. The probe includes a label that allows separation of the labeled microorganism. The probe is hybridized to the rRNA of the microorganism and the labeled target microorganism is separated from the unlabelled microorganisms.

The DNA can be recovered from the separated microorganism and portions thereof can be cloned into culturable microorganisms to synthesize the gene product.

### DETAILED DESCRIPTION OF THE INVENTION

The present invention comprises methods and devices for identifying and isolating unculturable microorganisms from a collection of microorganisms. The methods involve the use of a labeled nucleotide probe that is complementary to a variable region of the rRNA of the target microorganism. The probe is preferably complementary to a portion of a variable region of the 16S rRNA. The probe is hybridized to the rRNA of the target microorganism and the thus labeled microorganisms are separated from the collection of microorganisms using a technique based on the label, such as fluorescence activated cell separation (FACS), when the label is a fluorescent marker.

Definitions

The following terms, as used in this disclosure and claims, are defined as:

The term nucleotide refers to a subunit of a nucleic acid consisting of a phosphate group, a 5′ carbon sugar, and a nitrogen containing base. In RNA the 5′ carbon sugar is ribose. In DNA, it is a 2-deoxyribose. The term also includes analogs of such subunits.

The term nucleotide polymer refers to at least two nucleotides linked by phosphodiester bonds, also termed an oligonucleotide.

The term nucleic acid probe refers to a single stranded nucleic acid sequence that will combine with a complementary single stranded target nucleic acid sequence to form a double-stranded molecule (hybrid). A nucleic acid probe may be a nucleotide polymer.

The term hybrid refers to the complex formed between two single stranded nucleic acid sequences by Watson-Crick base pairings or non-canonical base pairings between the complementary bases.

The term hybridization refers to the process by which two complementary strands of nucleic acids combine to form double stranded molecules (hybrids).

The term complementarity refers to a property conferred by the base sequence of a single strand of DNA or RNA which may form a hybrid or double stranded DNA:DNA, RNA:RNA or DNA:RNA through hydrogen bonding between Watson-Crick base pairs on the respective strands. Adenine (A) usually complements thymine (T) or uracil (U), while guanine (G) usually complements cytosine (C).

The term probe specificity refers to characteristic of a probe that describes its ability to distinguish between target and non-target sequences. The specificity is dependent on sequence and assay conditions. Probe specificity may be absolute (i.e. the probe is able to distinguish between target organisms and any nontarget organisms), or it may be functional (i.e. the probe is able to distinguish between the target organism and any other organism normally present in a particular sample). Many probe sequences can be used for either broad or narrow specificity depending on the conditions of use.

The term variable region refers to nucleotide polymer which differs by at least one base between the target organism and nontarget organisms contained in a sample.

The term conserved region refers to a region which is not variable.

The term target sequence refers to the nucleic acid sequence to be labeled with the probe.

The term unculturable refers to those microorganisms that cannot be grown in pure cultures by known means.

The term detection refers to a method where analysis or viewing of the labeled target nucleic acid is possible visually or with the aid of a device. Such devices include microscopes, FACS devices, and fluorometers.

I. Methods of Isolating Unculturable Microorganisms

The target unculturable microorganism may be known beforehand and the methods described herein can be used to detect the microorganism in a sample and isolate the microorganism from the sample. Alternatively, the methods can be used to determine if a particular unculturable microorganism is contained within a sample.

In another embodiment of the invention, the methods described herein can be used to identify the unculturable microorganisms contained within a sample, and to select which of those microorganisms to isolate from the sample. The methods can then be used to identify and isolate the target microorganisms.

In a further step, the methods described herein can be used to isolate DNA from unculturable microorganisms isolated according to the methods described herein.

A. Identification of Unculturable Microorganisms in the Sample

In one embodiment of the present invention, the rRNA sequence of the target microorganism is known beforehand. It might also be known beforehand that the target microorganism is contained within the sample, or it might be desired to use the probe designed as taught herein to determine if the target microorganism is within the sample.

In another embodiment of the present invention, the unculturable microorganisms in the sample are identified using the technique described hereinafter. The sample can be from a variety of sources, such as from an environmental source or a biological source. For example, soil and water samples contain many unculturable microorganisms. The human gut also has been reported to contain many unculturable microorganisms. As a first step in determination of which of the microorganisms in the sample are unculturable, the DNA corresponding to the 16S rRNA of all of the microorganisms in the sample are isolated and sequenced. The DNA can be isolated from the collection of microorganisms and purified by methods known to those skilled in the art. Such methods for isolating DNA include a variety of techniques to disrupt the cell and liberate DNA into solution. Thereafter, the DNA is separated from the protein which is solubilized along with the DNA. The methods taught by Zhou et al., in “DNA Recovery from Soils of Diverse Composition”, Applied and Environmental Microbiology, 62(2):316-322 (February 1996) can be used for extraction and purification of DNA. Generally, this method involves extraction with a buffer at pH 8.0, 1.5 M NaCl, a detergent, hexadecylmethylammonium bromide (CTAB) and proteinase K (10 mg/ml). Another detergent treatment follows and then extraction with chloro/formisoamyl alcohol. The aqueous phase is recovered by centrifugation and precipitated with isopropanol. A pellet of crude nucleic acids is obtained by centrifugation. An RNase can be added during the extraction to remove residual RNA contamination. This crude sample is further purified using a DNA purification column such as a Magic PCR Purification Column (Promega, Madison, Wis.).

It may be necessary to use PCR amplification to increase the numbers of the DNA prior to sequencing. PCR can be performed following the techniques in “Techniques for Microbiol Ecology” Burlage, R. S. et al. (eds), Ch. 13, “Molecular Techniques”, Burlage, R. S., Oxford University Press, New York (1998). Generally, primers for conserved regions of the 16S rRNA gene are used to retrieve fragments of the nucleic acid. Suitable primers are known, as descibed in Chandler, D. P. et al., FEMS Microbiology Ecology 23:131-144 (1997). This reference also describes cloning and sequencing techniques that can be used. Once amplified, the DNA is cloned into plasmid vectors and transformed into an E. coli strain. From there the colonies are picked, the plasmids are isolated by a fast method, and the clones are screened by cutting the insert DNA with several restriction enzymes. Those displaying unique band patterns are sequenced. Including convenient restriction sites on the ends of the primers helps this process greatly.

Alternatively, the rRNA could be isolated and used. In this case only one primer would start the PCR reaction, and create a double stranded form. After this step then both primers are effective in amplifying the nucleic acid, and the products are cloned as usual. Since protocols are worked out for DNA isolation, and they are easy to perform, the DNA is usually isolated and used.

There are several known methods for purification of rRNA that can be used. The use of the powerfully chaotropic salts of guanidinium to simultaneously lyse cells, solubilize RNA and inhibit RNases is described in Chirgwin et al, Biochem., 18:5294-5299 (1979). Other methods free solubilized RNA of contaminating protein and DNA by extraction with phenol at an acidic pH using chloroform to effect a phase separation, as discussed in D. M. Wallace, Meth. Enzym., 152:33-41 (1987).

As a second step in determination of which of the microorganisms in the sample are unculturable, the sequences of the variable regions are compared to sequence databases of known microorganisms. Analysis of rRNA sequences is available via the Ribosomal Database Project Department of Microbiology, Michigan State University as described in Maidak, B. L. et al., Nucleic Acids Res Jan. 1, 1999;27(1):171-3. This database includes over 9700 small subunit rRNA sequences. Those sequences that do not match known sequences can then be designated as originating from unknown, and likely unculturable, microorganisms. Comparisons of the sequences are made using computer programs such as those available from the Ribosomal Database Project.

Alternatively, as a second step in determination of which of the microorganisms in the sample are unculturable, the collection of microorganisms can be mass cultured by means known in the art, or by a variety of means known in the art. The rRNA of the cultured collection(s) are sequenced and compared against the sequences of the rRNA in the original sample. The rRNA of the unculturable microorganisms can then be identified as the rRNA present in the collection but not present in the cultured collection.

The collection of microorganisms can be cultured by, for example, the techniques described in Chandler, D. P. et al., FEMS Microbiology Ecology 23:131-144 (1997). The collection of microorganisms can be blended in 0.1% Na₄P₂O₇.10H₂O, pH 7.0 and spread-plated, in serial 10-fold dilutions, on 1% peptone-tryptone-yeast extract-glucose agar or optimal plate agar. After incubation at 22° C. for 2-3 weeks, the colonies can be removed and grown in 10% liquid peptone-tryptone-yeast extract-glucose agar. There are many other culturing techniques known to those skilled in the art that can be used. Preferably, techniques known to be most useful for the microorganisms suspected of being present in the sample will be used. For example, the culturing conditions could be adjusted to be favorable for iron reducers, methane utilizers, nitrogen fixers, ammonia oxidizers, photosynthesizers, and other types of bacteria. Culturing techniques that can be used are taught in “Methods for General and Molecular Bacteriology” Gerhadt, P. et al. (eds) American Society for Microbiology Press, Washington, D.C. (1994); and “Techniques for Microbiol Ecology” Burlage, R. S. et al. (eds), Oxford University Press, New York (1998). Most preferably, two or more culturing techniques known to be most useful for the microorganisms suspected of being present in the sample are used and the rRNA of the unculturable microorganisms are then identified as the difference between the rRNA of the collection and the rRNA of the cultured collections.

B. Selecting which Unculturable Microorganism to Target

As discussed above, in one embodiment of the present invention, the target microorganism is known beforehand and the designed probe is used to isolate the target microorganism and/or to determine if the target microorganism is contained within the sample.

In another embodiment, the unculturable microorganism to target can be selected on the basis of its variable region. Since the variable regions of the 16S rRNA are often diagnostic for a particular species it may be preferred to select an unknown or unculturable microorganism because it belongs to a particular species which has shown certain properties. For example, an unculturable microorganism might be selected because it has a 16S rRNA region commonly found in a bacillus.

C. Construction of the Probe

The probe is preferably complementary to the 16S rRNA, although probes complementary to the 23S and, less preferably, the 5S rRNA could alternatively be used. When the sequence of all or a part of the 16S rRNA of the target unculturable microorganism has been determined, the probe can be constructed. The probe is an oligonucleotide having a sequence complementary to the identified rRNA. Preferably the probe is complementary to the variable region of the identified rRNA. The probe preferably is complementary to at least 5 nucleotides of the identified rRNA, most preferably to at least about 10 nucleotides, and most preferably is complementary to between about 15 to 30 nucleotides of the identified rRNA. A probe having a shorter complementary region will hybridize to more contaminating sites.

Accordingly, the probe preferably is at least about 5 nucleotides in length, more preferably about 10 and most preferably about 15-30 nucleotides in length. The probe is more likely to hybridize with the correct target nucleic acid if it has a longer complementary region but also may have difficulty entering the cell. In some cases it may be preferable or necessary to use a longer probe that will target more contaminating nucleic acids and then re-probe the detected nucleic acids with a more specific probe. It is preferable that the complementarity of the probe and the target be exact although the probe may still function if it contains one or two mismatched nucleotides.

U.S. Pat. Nos. 5,928,864 and 5,693,468 to Hogan et al. provide lengthy discussions of the factors associated with hybridization efficacy. The effects of these factors can be adjusted as discussed therein to provide a probe and hybridization conditions effective to label the target nucleic acid at sufficient quantities and yet not label contaminating nucleic acids.

The oligonucleotide is made using an automated DNA synthesizer as known by those skilled in the art. The probe may be composed of either RNA or DNA. Single stranded DNA is preferably used, and can be custom made by commercial suppliers.

The probe is labeled with a marker that will, preferably, allow the cell to be separated from the cells that do not contain hybridized, labeled rRNA. Many methods for labeling ligands known by those of skill in the art are suitable for purposes of the method of the present invention. For example, a magnetic marker can be used, that will allow the target, hybridized cells to be isolated using magnetic means. Digoxigenin-11-dUTP (Boehringer Mannheim, Indianapolis, Ind.) or biotin-16-dUTP (Boehringer Mannheim, Indianapolis, Ind.) can be incorporated into nucleic acid probes by nick translation (Rigby, et al., J. Mol. Biol., 113: 23, 1977) or random priming (Feinberg and Vogelstein, Anal. Biochem., 132:6-13,1983). Detection and separation of compounds containing biotin can be accomplished by incubating with avidin conjugated to the fluorophore of choice. Detection of digoxigenin-labeled nucleotides can be done with an anti-digoxigenin antibody conjugated to FITC, Texas Red, rhodamine or any other fluorophore. Monoclonal antibodies are often detected with secondary antibodies conjugated to the fluorophore of choice.

In a preferred embodiment, the marker comprises a fluorescent molecule that will allow the labeled cell to be separated using fluorescence-activated cell sorting (FACS). A fluorescent marker such as 2′,7′-difluorofluorescein (Oregon Green® 488), available from Molecular Probes, Inc. (Eugene, Oreg.) can be used. Other fluorescein analogs or substitutes can also be used, such as Rhodamine Green and Texas Red-X, also available from Molecular Probes, Inc. (Eugene, Oreg.).

Generally, amine-derivatized oligonucleotides, having an amine group on the 5′ end, can be labeled with an amine-reactive derivative of the fluorescent marker, such as a succinimidyl ester of the fluorescent marker. The amine-reactive fluorophore preferably contains aminohexanoic spacers to reduce the label's interaction with the oligonucleotide and enhance its accessibility to secondary detection reagents. Oregon Green 488, Rhodamine Green and Texas Red-X conjugates of dUTP and the Rhodamine Green conjugate of UTP are commonly used in nucleotide probes.

D. Hybridization

Solution or immobilized hybridization can be used. In general, in solution hybridization both the probe and the sample containing the target nucleic acid are in solution. In immobilized hybridization, on the other hand, the sample containing the target nucleic acid is immobilized on a solid support.

In situ hybridization is preferably used in the invention. In general, the labeled probe is contacted with cells that are fixed to be permeable to oligonucleotides. The labeled probe thus enters the cell and hybridizes to the complementary rRNA inside the cell.

Preferably, the sample is first subjected to treatments to remove contaminants. For example, the bacteria in a soil sample can be extracted by vortexing the sample at high speed in PBS and sodium pyrophosphate (10 mM). The bacteria can be washed.

The probe and the sample must be contacted under conditions where the probe can enter the bacterial cells in the sample but where the nucleic acids within the cell do not exit the cell. This can be accomplished by permeabilizing, or fixing, the cells, such as by treating the cells with 3% paraformaldehyde at 4° C. for a number of hours, depending on the nature of the sample (between about 3 and 30 hours). Fixed cells can be stored at −20° C. for a number of days.

Methods of hybridization are well known in the art. For example, Thomas J.-C. et al., Cytometry 27: 224-232 (1997) teaches a method of hybridization of whole cells that can be used in the present invention. That method involves fixing and permeabilizing the cells, such as with paraformaldehyde as described above, followed by suspension in a hybridization buffer at elevated temperature with the labelled probe. Mineral oil or other compounds can be added to minimize evaporation. Another method that can be used is described in “Techniques for Microbiol Ecology” Burlage, R. S. et al. (eds), Ch. 13, Burlage, R. S., “Molecular Techniques”, pages 319-22, Oxford University Press, New York (1998). The fixed cells are washed and suspended in a solution containing a non-ionic detergent and then dehydrated by repeated exposure to 50%, 80%, then 98% ethanol. The cells are then contacted with the hybridization probe in a hybridization buffer at the desired hybridization temperature. The optimal temperature is dependent upon the oligonucleotide and the dissociation temperature of the probe and target rRNA.

E. Separating the Labeled Microorganisms

After the targeted microorganisms are labeled with the probe, they are separated from the other cells. The method of separation to be used will depend upon the type of label that was used. Fluorescence-activated cell sorting (FACS) as described in Wallner, G. et al., Cytometry 14: 136-143 (1993), and Wallner, G. et al., Appl. Envir. Micro. 63: 4223-4231 (1997) is preferably used in the methods of the present invention. In this method, a FACStar PLUS flow cytometer (Becton Dickinson (BD), Mountain View, Calif.), equipped with two argon ion lasers, (Innova; Coherent, Palo Alto, Calif.) is used to measure forward angle light scatter (FSC), right angle light scatter (SSC), and fluorescence of the microbial cells. These parameters are acquired as pulse height signals (four decades in logarithmic scale) in list mode for 5,000 or 10,000 events at a rate of about 200 cells per second. The samples are analyzed using the standard stream-in-air configuration, usually triggered on FSC as threshold parameter. The lasers are tuned to the wavelength of the fluorescent label at a power output of 500 mW each. The label(s) are triggered at the appropriate wavelength and emission is measured with an appropriate wavelength band pass filter.

The recorded list-mode data files can be transferred by FASTFILE-software (BD) to an IBM-compatible PC-system. Data analysis and graphics can be performed using the DAS-software package (DAS=Data Analysis Software (DAS V 4.03), designed by W. Beisker.

For sorting, the method taught by Wallner, et al, “Flow Sorting of Microorganisms for Molecular Analysis” Appl. Envir. Micro. 63: 4223-4231 (1997) can be used. In this method, the drop drive frequency is set at approximately 27 kHz, 3 drops are simultaneously deflected, and droplet delay is set between 12 and 15. For sorting, 0.1% sodium chloride is used as sheath fluid. Sort criteria are defined by drawing polygonal gates in bivariate histograms (dot plots of the two most informative flow cytometric parameters) with the Lysis II software package (Becton Dickinson). In order to check if the selected populations really included the desired type of cells, the gated cells can be first sorted directly onto microscopic slides. After microscopic confirmation of the purity and correctness of cell sorting, sorted cells can be collected in sterile 1.5-ml reaction tubes for PCR, if necessary.

F. Removal of the Cellular DNA

The above methods may result in the capture of enough cells to allow direct DNA cloning, which is possible if there is about one microgram of DNA. However, if enough cells are not directly captured, the DNA can be extracted from the sorted cells and amplified by PCR using a set of random primers. The amplification products can then be cloned and sequenced.

The sequences can be added to databases and the transcription products determined.

IV. Methods of Using the Microorganism that Is Identified and Isolated

The DNA from the microorganisms that are labeled and separated can be isolated through means known in the art. The DNA can be sequenced and DNA of interest can be cloned into culturable microorganisms to synthesize the gene product. These microorganisms undoubtedly have undiscovered enzymes that would be useful for environmental, medical, or industrial applications medical technology, bioremediation of hazardous wastes, chemical and biological warfare detection, and industrial fermentation, for example.

All of the DNA sequences can be contributed to developing databases of DNA.

### EXAMPLES

The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

### Example 1

Detection of Soil Bacteria by in situ rRNA Hybridization.

Soil from the Dover Air Force Base (Dover, Del.) was collected for analysis. This soil was believed to contain an unculturable bacterium, called D1-38 strain. The rRNA of this bacterium was previously sequenced. The rRNA sequence from this unculturable bacterium was used to design two fluorescent oligonucleotide probes (JZ1 and JZ2; see Table 1) for use in locating and sorting the bacteria from the soil using the flow cytometer. A probe complementary to a 16S rRNA region conserved for all eubacteria was also used as a control. F indicates the fluorescein label site.

TABLE 1
Sequences of Fluorescently Labeled
Oligonucleotide rRNA Probes.

| | | |
|---|---|---|
| Name of Probe | Sequence (5′-3′) | Specificity |

| | | |
|---|---|---|
| EUB | (SEQUENCE ID NO: 1) | rRNA of |

| | |
|---|---|
| F GCT GCC TCC CGT AGG AGT | eubacteria |

| | | |
|---|---|---|
| +TC,1JZ1 | (SEQUENCE ID NO: 2) | D1-38 rRNA |

| | |
|---|---|
| F TCG CGC TTG AAG AGG CCC | sequence |
| CGC CC | |

| | | |
|---|---|---|
| +TC,1JZ2 | (SEQUENCE ID NO: 3) | D1-38 rRNA |

| | |
|---|---|
| F CCC GGG CTA ACA TCC CGG | sequence |
| GAG AG | |

About 10 g of a soil from a field site in Delaware (thought to be the site of the original D1-38 strain) was converted to a slurry by the addition of 90 ml of sterile 0.1% sodium pyrophosphate in water. It was vortexed three times for 30 seconds each time with 2 minute intervals. Then the slurry was incubated at 4° C. overnight with continuous gentle agitation. The large particles settled out after 30 minutes of standing at room temperature. This enabled decanting of the liquid into a new tube. This tube was centrifuged at 300 g for 5 minutes, and then 1000 g for 10 minutes, to separate the finer particles from the cells. The cells in suspension were decanted into a fresh tube filtered through a 20 micrometer filter set over a 8 micrometer cellulose filter. The cells were centrifuged at top speed in a Sorvall centrifuge at 4° C. for 10 minutes to form a concentrated cell pellet. The liquid was poured off and the cell pellet was resuspended in 3 ml of 3% fresh paraformaldehyde in PBS for 90 minutes at room temperature. The cells were spun down at top speed again and washed in 10 ml PBS. Finally the cells were resuspended in 2 ml PBS.

The cell suspension was divided into equal aliquots for the hybridization procedure. Each aliquot was approximately 200 microliters. The aliquot was spun down in a microfuge at top speed and washed once with 1 ml of PBS, then resuspended in 250 microliters of hybridization solution. (0.9 M NaCl, 20 mM Tris-HCl, pH 7.2; 0.01% SDS). The probe was added at 4 ng per microliter of hybridization solution. The mixture was incubated at 46° C. with gentle agitation for 3 hours. After this time the cells were spun down in the microfuge 6000 rpm for 2 minutes (room temperature) and the supernatent was discarded. 250 microliters of hybridization solution without probe was added and the mixture incubated at 46° C. for 30 minutes without agitation. The mixture was recentrifuged using the same conditions. The pellet was resuspended in 1 ml PBS and stored at 4° C. in the dark until needed. The cells were stained with ethidium bromide to visualize the bacteria. Ethidium bromide was used at a concentration of 1 mM.

The unprobed soil extraction samples showed a low level of autofluorescence in the area of the probe fluorescence region. The eubacteria were visible using the EUB fluorescent oligonucleotide, and the bacteria that hybridized with JZ1 or JZ2 were also visible.

### Example 2

Separation of the Probe-Labelled Bacteria.

After analysis of the samples for the presence of JZ1 or JZ2-positive cells, the JZ1 or JZ2-positive cells were sorted from the remaining soil/cell mixture using the sort feature of the flow cytometer.

The cells were sorted on a FACStar Plus fluorescent cell sorter (Becton Dickinson, San Jose, Calif.) equipped with a 488 nm Innova 90 argon laser set to 2 mW power. The bacterial cell solution was passed through the laser intercept at an average rate 600 events per second, or up to 1000 events per second, without loss of sorted sample. The Oregon Green fluorescence of the labeled bacteria is detected with a 520+/−15 nm bandpass filter, and the ethidium bromide is detected with a 640+/−30 nm bandpass filter. A sterile 0.1% sodium chloride solution is used as both the sheath fluid for sorting and as a collection fluid for the sorted bacterial cells. Collected cells were pelleted by centrifugation. A total of 5×10⁵ cells was sorted from approximately 4 ml of extracted cells. On average, 100,000 cells or more were collected for PCR analysis.

### Example 3

Extraction of DNA.

Total genomic DNA was extracted generally following the methods of Zhou et al., Appl. Envir. Micro. 62: 316-322 (1996). The sorted cells were pelleted in a microfuge for 20 minutes at top speed. The supernatent was discarded and the cells resuspended in 300 microliter of DNA extraction buffer (0.1 M phosphate buffer, pH 8.0; 0.1 M EDTA; 0.1 M Tris-HCl, pH 8.0; 1.5 M NaCl; 1% CTAB). The cells were freeze-thawed three times between −70° C. and room temperature (about 24° C.). Next 1.15 microliter of proteinase K (stock of 10 mg/ml) and 33 microliter of 20% SDS was added, the solution mixed well and then incubated at 65° C. for two hours with occasional mixing. After this time the cells were centrifuged at 10,000 rpm in a Sorvall centrifuge for 10 minutes to pellet the cell debris. The supernatant was collected in a fresh tube. An equal volume of chloroform was added and thoroughly mixed, after which the solution was centrifuged at 5500 rpm for 20 minutes to separate the two phases. The supernatent layer was drawn off into a new tube and 0.6 volumes of isopropanol was added and inverted several times to mix. This was stored overnight at −70° C. to precipitate DNA. The tube was spun at 11600 rpm for 25 minutes to collect the precipitated DNA. The supernatant was drawn off, the pellet was washed in a small amount of ethanol and then recentrifuged as before, and the ethanol poured off. The pellet was allowed to dry and was then dissolved in 50 microliter sterile distilled water. Later the sample was brought to a final volume of 200 microliters and column purified as described below.

A Magic PCR Purification Columns (Promega, Madison, Wis.) and a company-modified procedure for the purification of bacterial genomic DNA were used to remove residual humic acids and other PCR-inhibitory substances. PCR reactions with 1 μl of sorted bacterial DNA contained 2 μl 10×PCR buffer (500 mM KCl, 100 mM Tris-HCl (pH 9.0) and 1% Triton X-100), 2 μl 25 mM MgCl₂, 0.8 μl 25 mM dNTP mixture, 0.2 μl Taq polymerase (Perkin Elmer/Promega), and 20 pmol of each primer. 1% BSA was also added to some PCR reactions depending upon the primer set.

The nucleotide sequences of the primers, shown in Table 2, are universal primers for the conserved regions of the 16S gene sequence. The reaction was brought to 20 μl with sterile distilled water and overlaid with mineral oil. The reactions were processed 35 cycles (1 minute at 94° C., 1 minute at 48° C., and 2 minutes at 72° C.) followed by a 10 minute final extension. The reactions were analyzed by agarose gel electrophoresis. Reactions containing correct size PCR products were cloned into PCR 2.1 vector (Invitrogen). Clones containing inserts were initially selected using blue/white screening with IPTG (isopropyl-β-D thiogalactopyranoside) and X-gal (5-bromo-4-chloro-3-indolyl-β-D-5 galactoside), and analyzed for insert size by PCR with a TA PCR primer pair. Plasmids were extracted and purified with the Sephaglas purification kit (FMC). Partial sequences were determined using a Perkin Elmer automated DNA 373 sequencer using both cycle sequencing and dye termination techniques. Sequence analysis was performed using Blast! (Altshul et al., J. Mol. Biol. 215: 403-410 (1990)).

TABLE 2
PCR Primer Pairs to rRNA Sequences

| | | |
|---|---|---|
| PCR | | |
| Primer | | |
| pair | Sequence (5′-3′) | Specificity |

| | | |
|---|---|---|
| fd1 + | (SEQUENCE ID NO: 4) | All bacterial |

| | | |
|---|---|---|
| rp1 | AGA GTT TGA TCC TGG CTC AGC | 16S rDNA |
| | ACG GTT ACC TTG TTA CGA CTT | sequences |

| | | |
|---|---|---|
| EUB + | (SEQUENCE ID NO: 5) | Eubacterial DNA |

| | | |
|---|---|---|
| Y1540R | GCT GCC TCC CGT AGG AGT AAG | sequences |
| | GAG GTG ATC GAG CC | |

The PCR reactions with the DNA obtained from the sorted fluorescently labeled bacteria produced two clones of the correct PCR product size. These PCR reactions were cloned and sequenced; each produced the same rRNA sequence. When this sequence was used to search against all the sequences in the GenBank, no exact matches were found.

Sequencing confirmed that the strain from which the sequence was obtained is a soil bacterial strain. However, it was not similar to either E. coli rRNA or the 16S sequence of the D1-38 strain. There is no guarantee that the D1-38 strain was present in the soil sample, and the collected cells may represent the next closest species in terms of DNA homology. Based on the high percentage of homology with similar sequences, the rRNA obtained from the PCR products represents a new species in the genus Pseudomonas.

The above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patents, patent applications, and publications, are incorporated herein by reference.

What is claimed is:

1. A method of isolating a target unculturable microorganism from a collection of microorganisms, comprising the steps:
obtaining a probe for the target microorganism, wherein the probe comprises a nucleic acid sequence complementary to at least a portion of the variable region of the rRNA of the target microorganism;
attaching a label to the probe, wherein the label enables separation of any labeled substrate;
in situ hybridizing the probe to the rRNA of the target microorganism; and
separating the labeled target microorganism from the unlabelled microorganisms.

2. The method of claim 1, wherein the step of obtaining a probe for the target microorganism comprises the steps:
sequencing the rRNA from the collection of microorganisms;
culturing the collection of microorganisms;
sequencing the rRNA from the cultured collection of microorganisms;
comparing the rRNA sequences of the collection of microorganisms to the rRNA sequences of the cultured collection of microorganisms;
determining the rRNA sequences of the unculturable microorganisms as the difference of the rRNA sequences of the collection of microorganisms and the rRNA sequences of the cultured collection of microorganisms;
selecting one of the unculturable microorganisms as the target microorganism; and
constructing a nucleic acid sequence complementary to at least a portion of the variable region of the rRNA of the target microorganism.

3. The method of claim 1, wherein the step of obtaining a probe for the unculturable microorganism comprises the steps:
sequencing the rRNA from the collection of microorganisms;
comparing the rRNA sequence from the collection of microorganisms to at least one database of sequences of known rRNA from culturable microorganisms;
determining the rRNA sequences of the unculturable microorganisms as the difference of the rRNA sequences of the collection of microorganisms and the rRNA sequences from the at least one database;
selecting one of the unculturable microorganisms as the target microorganism; and
constructing a nucleic acid sequence complementary to at least a portion of the variable region of the rRNA of the unculturable microorganism.

4. The method of claim 1, wherein the probe comprises between about 5 and 30 nucleotides and is complementary to between about 5 and 30 nucleotides of the variable region of the rRNA of the target microorganism.

5. The method of claim 4, wherein the probe comprises between about 15 to 30 nucleotides.

6. The method of claim 1, wherein the label comprises a fluorescent molecule and wherein the step of separating the labeled microorganism comprises using fluorescence activated cell separation.

7. The method of claim 1 wherein the collection of microorganisms is from a soil sample, a biological sample, or a water sample.

8. The method of claim 1, wherein the rRNA is the 16S rRNA.

9. The method of claim 1, wherein the target microorganism is selected because it has a rRNA region typical to a particular type of microorganism.

10. A method of isolating DNA from an unculturable microorganism comprising the method of claim 1 and further comprising isolating the DNA from the separated microorganism.

11. A gene product produced from the DNA isolated according to the method of claim 10.

12. A method for identifying a target unculturable microorganism in a collection of microorganisms, comprising:
obtaining a probe for the target microorganism, wherein the probe comprises a nucleic acid sequence complementary to at least a portion of the variable region of the rRNA of the target microorganism;
attaching a detectable label to the probe;
hybridizing the labeled probe to the rRNA of the target microorganism; and
detecting the detectable label.

13. The method of claim 12, wherein the step of obtaining a probe for the target microorganism comprises the steps:
sequencing the rRNA from the collection of microorganisms;
culturing the collection of microorganisms;
sequencing the rRNA from the cultured collection of microorganisms;
comparing the rRNA sequences of the collection of microorganisms to the rRNA sequences of the cultured collection of microorganisms;
determining the rRNA sequences of the unculturable microorganisms as the difference of the rRNA sequences of the collection of microorganisms and the rRNA sequences of the cultured collection of microorganisms;
selecting one of the unculturable microorganisms as the target microorganism; and
constructing a nucleic acid sequence complementary to at least a portion of the variable region of the rRNA of the target microorganism.

14. The method of claim 12, wherein the step of obtaining a probe for the unculturable microorganism comprises the steps:
sequencing the rRNA from the collection of microorganisms;
comparing the rRNA sequence from the collection of microorganisms to at least one database of sequences of known rRNA from culturable microorganisms;
determining the rRNA sequences of the unculturable microorganisms as the difference of the rRNA sequences of the collection of microorganisms and the rRNA sequences from the at least one database;
selecting one of the unculturable microorganisms as the target microorganism; and
constructing a nucleic acid sequence complementary to at least a portion of the variable region of the rRNA of the unculturable microorganism.

15. The method of claim 12, wherein the probe comprises between about 5 and 30 nucleotides and is complementary to between about 5 and 30 nucleotides of the variable region of the rRNA of the target microorganism.

16. The method of claim 15, wherein the probe comprises between about 15 to 30 nucleotides.

17. The method of claim 12, wherein the detectable label comprises a fluorescent molecule.

18. The method of claim 12 wherein the collection of microorganisms is from a soil sample, a biological sample, or a water sample.

19. The method of claim 12, wherein the rRNA is the 16S rRNA.

20. The method of claim 12, wherein the target microorganism is selected because it has a rRNA region typical to a particular type of microorganism.
